SEQUENCE LISTING (3 sequences)

SEQ ID NO: 1 - DNA, 32 bp, Artificial Sequence
  Description of Artificial Sequence Forward primer
acttggatccatccatctccgacgagatgagt

SEQ ID NO: 2 - DNA, 30 bp, Artificial Sequence
  Description of Artificial Sequence Reverse primer
gctgttatgacctctacttacttaagttca

SEQ ID NO: 3 - DNA, 272 bp, Escherichia coli
acttggatccatctccgacgagatgagtgtaaaaatcgtgctgtcgattaacctttcgcctgttgccgccgttgtcgatttactggcaatcacggcattaagtgggtgatttgcttcacatctcgggcattttcctgcaaaaccatacccttacgaaaagtacggcattgataatcattttcaatatcatttaattaactataatgaaccaactgcttacgcggcattaacaatcggccgcccgacaatactggagatgaatgaattcaagt

(19) United States
(12) Patent Application Publication
(10) Pub. No.: US 2003/0134348 A1
(43) Pub. Date: Jul. 17, 2003

(54) Recombinant bioluminescent bacteria Escherichia coli DH5@/pSodaLux (EBHJ1) [KCTC 10098BP] for monitoring oxidative stress

(76) Inventors: Man Bock Gu, Kwangju (KR); Hyun Joo Lee, Seoul (KR)

Correspondence Address: PENNIE AND EDMONDS, 1155 AVENUE OF THE AMERICAS, NEW YORK, NY, 100362711

(21) Appl. No.: 10/288,107

(22) Filed: Nov. 5, 2002

(30) Foreign Application Priority Data
Nov. 17, 2001 (KR) 2001-0071591

(51) Int. Cl. C12Q 1/18; C12N 1/21; C12N 15/74

(52) U.S. Cl. 435/32; 435/252.33; 435/320.1

(57) ABSTRACT

The present invention relates to recombinant bioluminescent bacteria Escherichia coli DH5@/pSodaLux (EBHJ1) [KCTC 10098BP] for monitoring oxidative stress. Recombinant vector pSodaLux including the SOD (superoxide dismutase) promoter gene is introduced to Escherichia coli to produce transformed recombinant bioluminescent bacteria Escherichia coli DH5@/pSodaLux (EBHJ1). These bacteria are sensitive in monitoring oxidative stress, which is fatal to living organisms.

### BACKGROUND OF THE INVENTION

1. Field of the Invention

The present invention relates to recombinant bioluminescent bacteria Escherichia coli DH5@/pSodaLux (EBHJ1) [KCTC 10098BP] for monitoring oxidative stress. Recombinant vector pSodaLux including the SOD (superoxide dismutase) promoter gene is introduced to Escherichia coli to produce transformed recombinant bioluminescent bacteria Escherichia coli DH5@/pSodaLux (EBHJ1). These bacteria are sensitive in monitoring oxidative stress, which is fatal to living organisms.

2. Description of the Prior Art

Most of the conventional bioluminescent bacteria emit certain lights at normal conditions. When they are exposed to toxic materials, the light emission reduces due to death or decrease of metabolism [Microtox™, Microbics Corp., Carlsbad, Calif., U.S.].

Oxidative stress is very harmful to microorganisms, plants and animals. It is because the reactive oxygen free radical transforms cell membranes, proteins and nucleic acids. Also in humans, it causes retrogressive diseases like Alzheimer's disease, diabetes and fatal diseases like cancers. Therefore, monitoring of natural and synthetic toxic materials causing oxidative stress is highly required.

Until now, the most common toxicity monitoring using bioluminescent bacteria is the Microtox method [Jennings et al., Water Res. 2001 October; 35(14): 3448-56]. However, this method determines toxicity by the extinction level of microorganisms exposed to toxic materials, specific reactivity like oxidative stress cannot be monitored by this method.

### SUMMARY OF THE INVENTION

The inventors developed recombinant bioluminescent bacteria Escherichia coli DH5@/pSodaLux (EBHJ1) [KCTC 10098BP] using promoter of SOD (superoxide dismutase), which is the most essential enzyme in protection against and recovery from oxidative stress. Using these bacteria, it is possible to monitor environmental toxicity causing oxidative stress easily and broadly.

Accordingly, an object of this invention is to provide recombinant bioluminescent bacteria Escherichia coli DH5@/pSodaLux (EBHJ1) [KCTC 10098BP] for monitoring oxidative stress.

### BRIEF DESCRIPTION OF THE DRAWINGS

FIG. 1 is the structure of recombinant vector pSodaLux and the electrophoresis (in 1% agarose gel) photograph of genes obtained by PCR.

FIG. 2 is a graph that shows bioluminescence of recombinant bioluminescent bacteria Escherichia coli DH5@/pSodaLux (EBHJ1) [KCTC 10098BP] and the control cell line (wild type).

FIG. 3 is a graph that shows bioluminescence of recombinant bioluminescent bacteria Escherichia coli DH5@/pSodaLux (EBHJ1) [KCTC 10098BP] when exposed to paraquat dichloride.

FIG. 4 is a graph that shows bioluminescence of recombinant bioluminescent bacteria Escherichia coli DH5@/pSodaLux (EBHJ1) [KCTC 10098BP] when exposed to hydrogen peroxide.

FIG. 5 is a graph that shows bioluminescence of recombinant bioluminescent bacteria Escherichia coli DH5@/pSodaLux (EBHJ1) [KCTC 10098BP] when exposed to potassium chromate.

FIG. 6 is a graph that shows bioluminescence of recombinant bioluminescent bacteria Escherichia coli DH5@/pSodaLux (EBHJ1) [KCTC 10098BP] when exposed to cadmium chloride.

FIG. 7 is a graph that shows bioluminescence of recombinant bioluminescent bacteria Escherichia coli DH5@/pSodaLux (EBHJ1) [KCTC 10098BP] when exposed to radioactive ray (γ-ray).

### DETAILED DESCRIPTION OF THE INVENTION

The present invention is characterized by recombinant bioluminescent bacteria Escherichia coli DH5@/pSodaLux (EBHJ1) [KCTC 10098BP] transformed by introducing the recombinant vector pSodaLux, which has the SOD (superoxide dismutase) promoter gene, to Escherichia coli and oxidative stress monitoring using these bacteria.

Hereunder is given a more detailed description.

The present invention relates to recombinant bioluminescent bacteria Escherichia coli DH5@/pSodaLux (EBHJ1) [KCTC 10098BP], which are transformed by genetic engineering, for monitoring of oxidative stress, which may be fatal to living organisms. These recombinant bioluminescent bacteria can be utilized in monitoring of oxidative stress for chemicals or radioactive materials.

The recombinant vector pSodaLux is produced by fusing promoter of the SOD gene, which participates in primary defense against and recovery from oxidative stress, with the LuxCDABE operon gene, which causes bioluminescence by stimulus without external requirements.

The recombinant vector pSodaLux was introduced to Escherichia coli DH5@. From the colonies expressed from the transformant having the recombinant vector pSodaLux, the one that emits light in proportion to oxidative stress most effectively was selected. These transformed recombinant bioluminescent bacteria Escherichia coli DH5@/pSodaLux (EBHJ1) were deposited in KCTC (Korean Collection for Type Cultures) of KRIBB (Korea Research Institute of Bioscience and Biotechnology) on Oct. 30, 2001. The Deposition No. is KCTC 10098BP.

Because the recombinant bioluminescent bacteria Escherichia coli DH5@/pSodaLux (EBHJ1) operates the SOD (superoxide dismatase) promoter in an environment that may cause oxidative stress and emits bioluminescence, they can be used to monitor toxicity easily and sensitively.

The following examples are to be illustrative of the present invention. However, they should not be construed as limiting the scope of this invention.

### EXAMPLE 1

### Production of Recombinant Vector for Monitoring Oxidative Stress

We obtained a 272 bp product (Sequence No.3) with 5′-primer (Sequence No.1: 5′-ACTTGGATCCATCCATCTCCGACGAGATGAGT-3′) and 3′-primer (Sequence No.2: 5′-GCTGT TATGA CCTCT ACTTA CTTAA GTTCA-3′) having BamHI and EcoRI restriction enzyme sites at the promoter part of the sodA gene from PCR using genomic DNA isolated from Escherichia coli [Quiagen DNA mini kit; Quiagen] as template. The 5′-primer was prepared from the leading part of an entire sodA gene promoter part. The 3′-primer was prepared to just before the initiation codon (ATG) of the sodA gene [NCBI AE000465]. The PCR was carried out 32 cycles under the condition of 30 s at 94° C., 30 s at 60° C. and 1 min at 72° C. This product was obtained by treating the vector pUCD615 [Rogowsky, et al. (J. Bacteriol., 169 (11) pp. 5101-512, (1987))] containing kanamycin and ampicillin resistant genes and not including promoter before the LuxCDABE operon gene of luminescent Vibrio fischeri with BamHI and EcoRI restriction enzymes. It was named as pSodaLux.

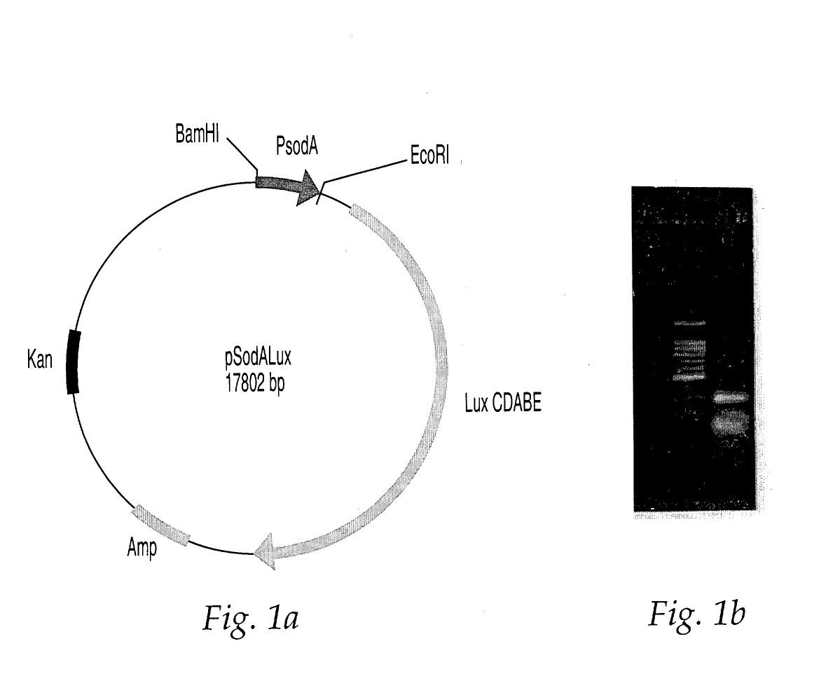

A product band of suitable size was obtained by carrying out PCR under the same condition using this vector pSodaLux as template and the same primer set. This product band was identified by electrophoresis in 1% agarose gel [FIG. 1].

### EXAMPLE 2

### Production of Recombinant Bioluminescent Bacteria

The vector pSodaLux produced in Example 1 was introduced to Escherichia coli (MAX Efficiency DH5@ Competent Cells; GIBCOBRL) to transform it. Cells having pSodaLux can survive due to action of the anti-kanamycin gene. Among the surviving cells, colonies that emit stable bioluminescence were selected. Among these colonies, those that emit light effectively in proportion to the oxidative stress level were selected.

These transformed recombinant bioluminescent bacteria Escherichia coli DH5@/pSodaLux (EBHJ1) were deposited in KCTC of KRIBB on Oct. 30, 2001 and Deposition No. KCTC 10098BP was assigned accordingly.

### EXAMPLE 3

### Bioluminescence Determination of Transformed Recombinant Bioluminescent Bacteria Escherichia coli DH5@/pSodaLux (EBHJ1) [KCTC 10098BP]

The bioluminescent bacteria Escherichia coli DH5@/pSodaLux (EBHJ1) were cultured in a shaking incubator of 30° C. at 250 rpm, in the LB medium [DIFCO, USA] containing 25 μg/ml of kanamycin [Sigma Co., USA] until the OD reaches 0.8 at 600 nm. Then, 2 ml of the culture was cultured for about 4 hr in 100 ml of LB medium. After adding 160 μl of these recombinant bioluminescent bacteria Escherichia coli DH5@/pSodaLux (EBHJ1) [KCTC 10098BP] in a 96-well plate [Microlite™; DYNEX Technologies, USA], 40 μl of toxic materials of the concentrations of 0, 0.03125, 0.0625, 0.125, 0.25, 0.5, 1, 2, 4, 8, 16, 32, 64, 128, 256, 512, 1024 and 2048 ppm were injected. The bioluminescence was measured at 30° C. with a 96-well luminometer [DYNEX Technologies, USA].

The bioluminescence is measured in BL (bioluminescence intensity). Increase in bioluminescence can be determined by comparing the bioluminescence when toxic material was injected (sample bioluminescence) and that when no toxic material was injected (control bioluminescence). The RBL (relative bioluminescence) is calculated by the following Equation 1.

$$RBL = \frac{Biolu min escence when toxic material was injected}{Biolu min escence when no toxic material was injected } \qquad Equation 1$$

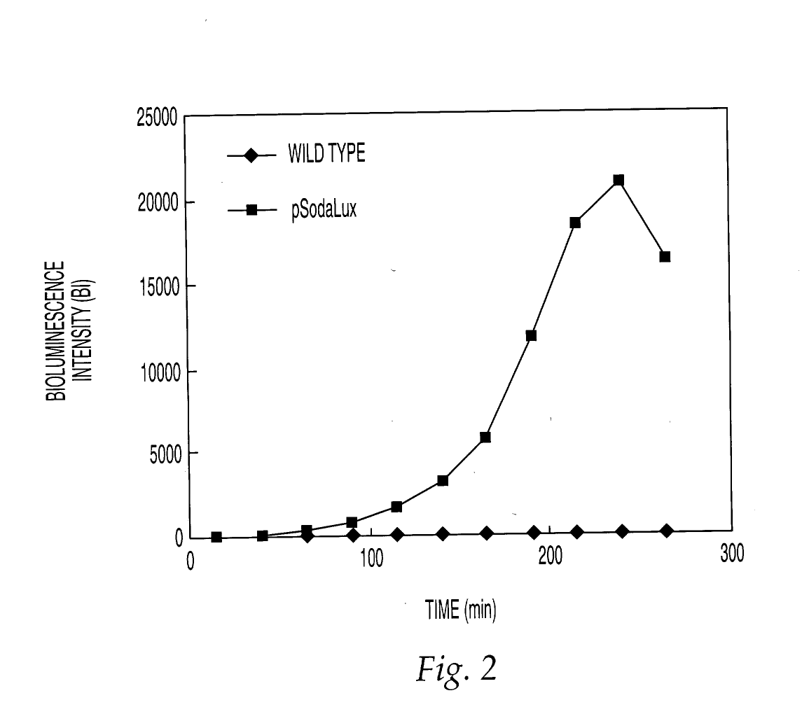

The recombinant bioluminescent bacteria Escherichia coli DH5@/pSodaLux (EBHJ1) [KCTC 10098BP] according to the present invention increases bioluminescence under oxidative stress. As shown in FIG. 2, they exhibit higher bioluminescence than the control cell line (wild type).

### EXAMPLE 4

### Determination of Oxidative Stress Monitoring of Recombinant Bioluminescent Bacteria Escherichia coli DH5@/pSodaLux (EBHJ1) [KCTC 10098BP] for Toxic Materials

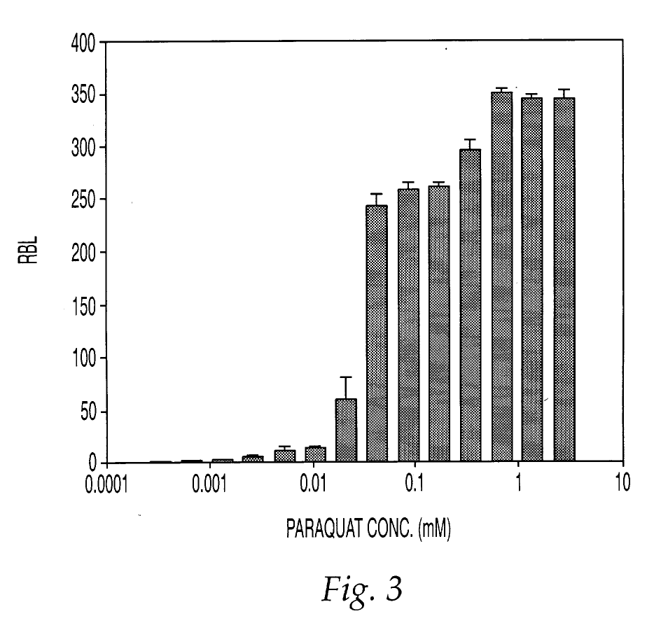

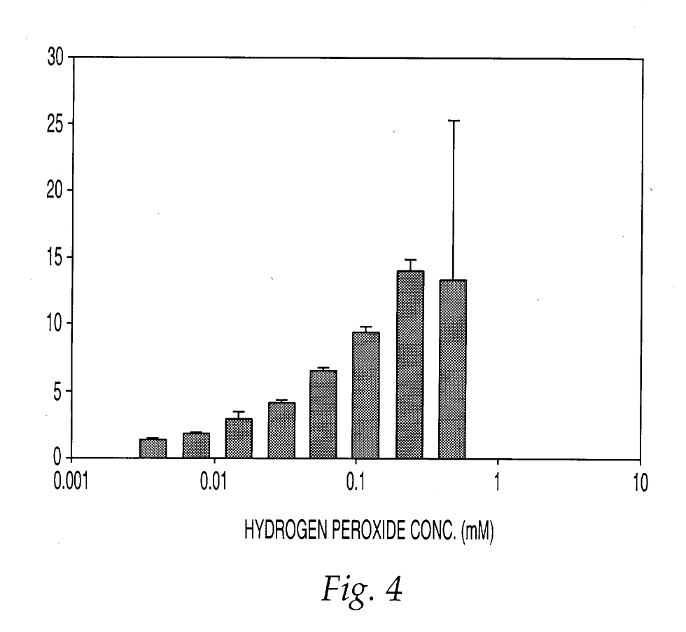

The recombinant bioluminescent bacteria Escherichia coli DH5@/pSodaLx (EBHJ1) [KCTC 10098BP] exhibited proportionally increasing bioluminescence for paraquat dichloride, which is best known as redox cycle agent (FIG. 3). Also, they exhibited substantial bioluminescence around 50 ppm, which is the concentration that causes little growth inhibition. And, they exhibited proportionally increasing bioluminescence for hydrogen peroxide, too (FIG. 4).

To sum up, it was possible to monitor oxidative stress sensitively using the recombinant bacteria Escherichia coli DH5@/pSodaLux (EBHJ1) of the present invention.

### EXAMPLE 5

### Determination of Oxidative Stress Monitoring for Heavy Metals

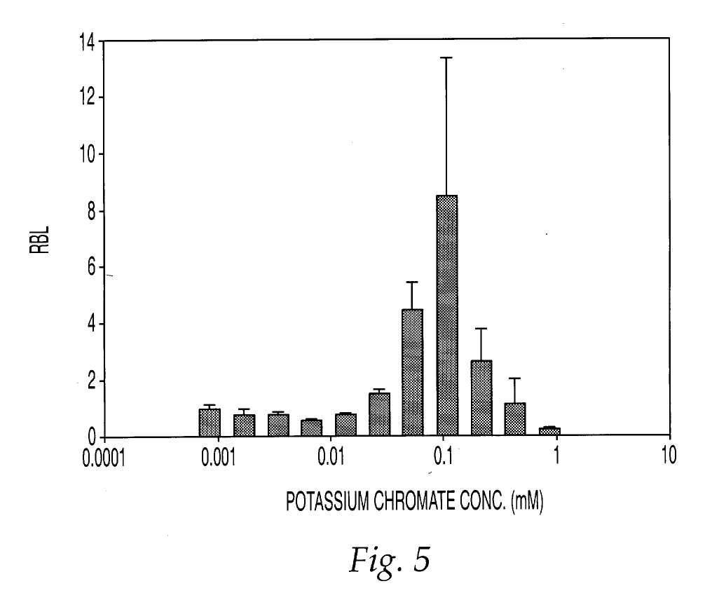

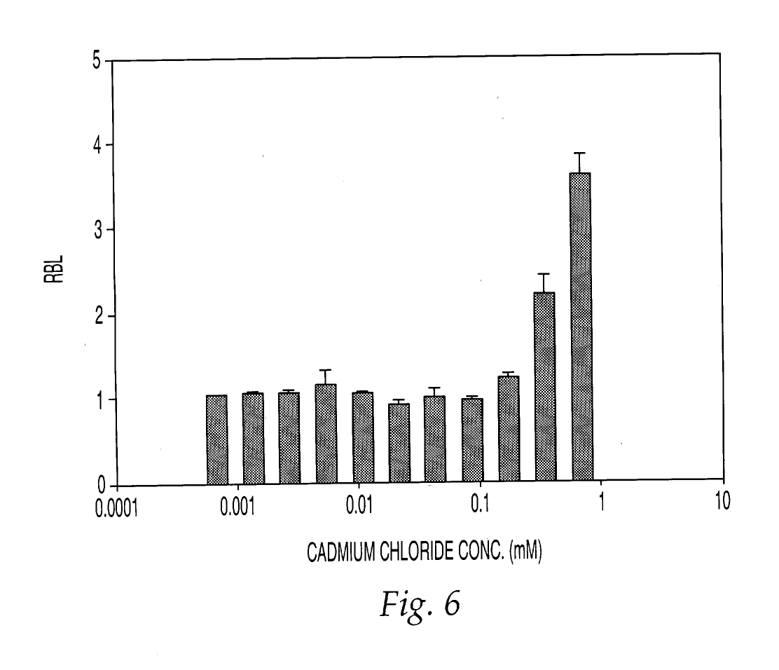

Bioluminescence of the recombinant bioluminescent bacteria Escherichia coli DH5@/pSodaLux (EBHJ1) [KCTC 10098BP] was measured for potassium chromate and cadmium chloride. As shown in FIGS. 5 and 6, the bioluminescence increased with the concentration of the heavy metals.

### EXAMPLE 6

### Determination of Oxidative Stress Monitoring for Reactive Oxygen

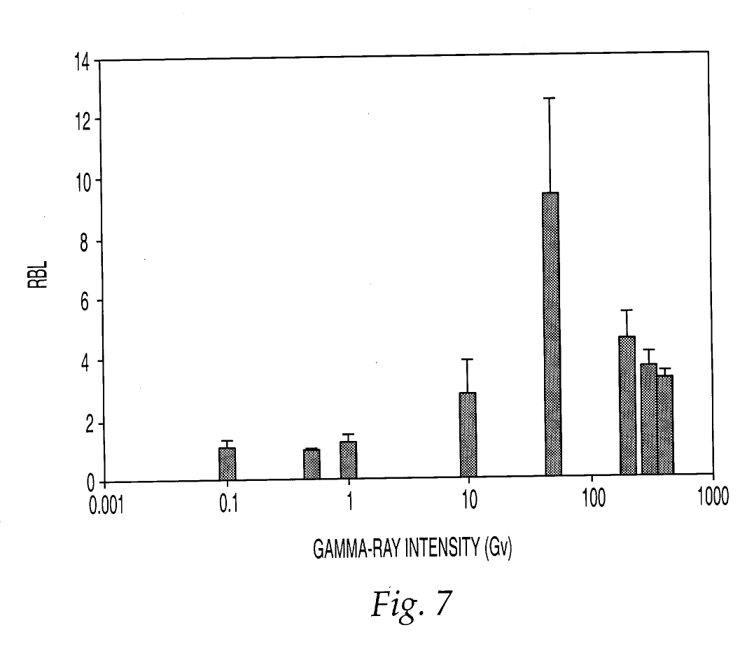

To investigate luminescence of the recombinant bioluminescent bacteria Escherichia coli DH5@/pSodaLux (EBHJ1) [KCTC 10098BP] due to reactive oxygen when exposed to radioactive rays, we illuminated γ-rays of 0.1-400 Gy. As shown in FIG. 7, the intensity of γ-ray is very closely related to the luminescence.

As explained above, the recombinant bioluminescent bacteria Escherichia coli DH5@/pSodaLux (EBHJ1) [KCTC 10098BP] according to the present invention can be utilized in monitoring of oxidative stress, which may be fatal to the ecosystem including human beings.

Also, they can be contribute to researches on identifying oxidative stress-related cell metabolism and diseases.

What is claimed is:

1. A recombinant vector pSodaLux that contains the SOD (superoxide dismutase) promoter gene.

2. Transformed recombinant bioluminescent bacteria Escherichia coli DH5@/pSodaLux (EBHJ1) [KCTC 10098BP] obtained by introducing the recombinant vector pSodaLux to Escherichia coli DH5@.

3. A method monitoring oxidative stress by utilizing the recombinant bioluminescent bacteria Escherichia coli DH5@/pSodaLux (EBHJ1) [KCTC 10098BP].
